(19) United States
(12) Patent Application Publication
(10) Pub. No.: US 2015/0320709 A1
(43) Pub. Date: Nov. 12, 2015

(54) METHODS AND COMPOSITIONS FOR FACILITATING WEIGHT LOSS BY ADMINISTRATION OF THYROID HORMONES

(71) Applicants: Thomas Najarian, Incline Village, NV (US); Carol N. Rowsemitt, San Luis Obispo, CA (US)

(72) Inventors: Thomas Najarian, Incline Village, NV (US); Carol N. Rowsemitt, San Luis Obispo, CA (US)

(21) Appl. No.: 14/568,428

(22) Filed: Dec. 12, 2014

Related U.S. Application Data
(63) Continuation of application No. 13/537,853, filed on Jun. 29, 2012, now abandoned.
(60) Provisional application No. 61/529,349, filed on Aug. 31, 2011.
(60) Provisional application No. 61/504,089, filed on Jul. 1, 2011.

(51) Int. Cl. A61K 31/197

(52) U.S. Cl. CPC A61K 31/197

(57) ABSTRACT

The present invention provides methods and compositions for facilitating weight loss in subjects who are undergoing a weight loss regimen by administration of thyroid hormones, for example, T₂ alone, T₂ in combination with T₃, T₂ in combination with T₃ and T₄, or T₃ in combination with T₄. In particular, subjects who have already experienced weight loss often are unable to continue to lose weight as the body experiences a famine response, i.e., hypothyroidism. By administration of the compositions of the present invention, for example, T₂ alone, T₂ in combination with T₃, T₂ in combination with T₃ and T₄, or T₃ in combination with T₄, subjects are able to return thyroid levels to normal and/or to reduce the levels of rT₃ to normal, so as to allow the body to continue to lose weight.

### RELATED APPLICATIONS

This application is a continuation of U.S. patent application Ser. No. 13/537,853, filed Jun. 29, 2012, which is related and claims priority to U.S. Provisional Application No. 61/504,089, filed Jul. 1, 2011 and U.S. Provisional Application No. 61/529,349, filed Aug. 31, 2011, the entire contents of each of which are hereby incorporated by reference herein.

### FIELD OF THE INVENTION

The present invention is directed to methods and compositions for facilitating weight loss by administration of thyroid hormones, including, for example, T₂ alone, T₂ in combination with T₃, T₂ in combination with T₃ and T₄, or T₃ in combination with T₄.

### SUMMARY OF THE INVENTION

In one aspect, the present invention is directed to a method for facilitating or continuing weight loss in a subject undergoing a weight loss regimen, the method comprising administering to the subject T₂, T₂ in combination with T₃, T₂ in combination with T₃ and T₄, or T₃ in combination with T₄, in an amount effective to increase the level of circulating thyroid hormone in the subject or to reduce the level of circulating rT₃ hormone to normal, thereby facilitating or continuing weight loss in the subject. In another aspect, the present invention is directed to a method for alleviating or preventing a thyroid abnormality in a subject undergoing a weight loss regimen, the method comprising administering to the subject T₂, T₂ in combination with T₃, T₂ in combination with T₃ and T₄, or T₃ in combination with T₄, in an amount effective to increase the level of circulating thyroid hormone in the subject or to reduce the level of circulating rT₃ hormone to normal, thereby alleviating or preventing a thyroid abnormality in the subject. For example, the thyroid abnormality may be selected from the group consisting of elevated diastolic blood pressure, low blood pressure, elevated lipid levels, coldness, cold extremities, fatigue, insomnia, muscle aches, joint aches, hair loss, constipation, depression and a combination thereof.

In various embodiments, the subject has a reduced level of circulating thyroid hormone prior to administration of the thyroid hormone, for example, as compared to the level of circulating thyroid hormone prior to starting the weight loss regimen. In certain embodiments, the subject has a reduced level of circulating T₃, circulating T₄ or a combination thereof. Alternatively or in combination, the subject may have an elevated level of circulating rT₃ prior to administration of the thyroid hormone.

In particular embodiments, the subject has experienced weight loss prior to administration of the thyroid hormone. For example, the subject may have lost at least 5, 10, 15, 20, 25, 30, 35, 40, 45, 50, 55, 60, 65, 70, 75, 80, 85, 90, 95, 100, 150 or 200 pounds prior to administration of the thyroid hormone. The subject may have experienced weight loss within 1, 2, 3, 4, 5, 6, 7, 8, 9, 10, 11, 12, 18 or 24 months prior to administration of the thyroid hormone. In certain embodiments, the weight loss regimen includes dieting, exercise, weight loss surgery and/or appetite control medication. In various embodiments, the subject has been undergoing a weight loss regimen for at least 1, 2, 3, 4, 5, 6, 7, 8, 9, 10, 11, 12, 18 or 24 months prior to administration of the thyroid hormone.

In one embodiment, the subject is administered T₂ alone. In another embodiment, the subject is administered T₂ in combination with T₃. In yet another embodiment, the subject is administered T₂ in combination with T₃ and T₄. In yet another embodiment, the subject is administered T₃ in combination with T₄.

In certain embodiments of the foregoing aspects of the invention, the subject is administered at least about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mcg of T₃ daily. In other embodiments, the subject is administered between about 5 and about 500 mcg, about 10 and about 400 mcg, about 15 and about 300 mcg, about 20 and about 200 mcg, about 25 and about 150 mcg, or about 50 and about 100 mcg of T₃ daily and, in a particular embodiment, between about 25 and about 150 mcg of T₃ daily.

In various embodiments, the subject is further administered T₄. For example, the subject may be administered at least about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mcg of T₄ daily. In other embodiments, the subject may be administered between about 5 and about 500 mcg, about 5 and about 400 mcg, about 5 and about 300 mcg, about 10 and about 200 mcg, about 10 and about 150 mcg, or about 50 and about 100 mcg of T₄ daily and, in a particular embodiment, between about 10 and about 150 mcg of T₄ daily.

In various embodiments, the subject is administered at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190 or 200 mcg of T₂ daily. In other embodiments, the subject is administered between about 0.5 and about 200 mcg, about 0.5 and about 150 mcg, about 0.5 and about 100 mcg, about 1 and about 100 mcg, about 1 and about 75 mcg, about 1.5 and about 75 mcg, about 1 and about 50 mcg, about 2 and about 50 mcg, about 1 and about 25 mcg, about 5 and 25 mcg, about 1 and about 10 mcg, or about 1 and about 5 mcg of T₂ daily.

In certain embodiments, T₃ and T₄ are administered in a ratio of about 50:1, about 45:1, about 40:1, about 35:1, about 30:1, about 25:1, about 20:1, about 15:1, about 10:1, about 9:1, about 8:1, about 7:1, about 6:1, about 5:1, about 4:1, about 3:1, about 5:2, about 2:1, about 3:2, about 1:1, about 2:3 or about 1:2. In other embodiments, T₃ and T₄ are administered in a ratio between about 50:1 and about 1:2, about 40:1 and about 1:2, about 30:1 and about 1:2, about 20:1 and about 1:2, about 10:1 and about 1:2, about 5:1 and about 1:2, about 5:1 and about 2:3, about 4:1 and about 1:2, about 4:1 and about 2:3, about 3:1 and about 1:2, about 3:1 and about 1:2, about 2:1 and about 1:2, about 2:1 and about 2:3, about 1:1 and about 1:2, or about 1:1 and about 2:3.

In various embodiments, at least one of T₂, T₃ and T₄ is administered in a sustained release composition. In certain embodiments, the combination of T₂ and T₃; T₂, T₃ and T₄; or T₃ and T₄ are provided in a single formulation, for example, a sustained release formulation.

In another aspect, the present invention provides a pharmaceutical composition for facilitating weight loss, comprising a combination of T₃ and T₄ in a ratio between about 50:1 and about 1:2, about 40:1 and about 1:2, about 30:1 and about 1:2, about 20:1 and about 1:2, about 10:1 and about 1:2, about 5:1 and about 1:2, about 5:1 and about 2:3, about 4:1 and about 1:2, about 4:1 and about 2:3, about 3:1 and about 1:2, about 3:1 and about 1:2, about 2:1 and about 1:2, about 2:1 and about 2:3, about 1:1 and about 1:2, or about 1:1 and about 2:3; and a pharmaceutically acceptable carrier. Optionally, the composition further includes T₂.

In yet another aspect, the present invention provides a pharmaceutical composition for facilitating weight loss, comprising T₃ and T₄; and a pharmaceutically acceptable carrier, for example, a sustained release composition. In certain embodiments, T₃ and T₄ are present in a ratio between about 50:1 and about 1:2, about 40:1 and about 1:2, about 30:1 and about 1:2, about 20:1 and about 1:2, about 10:1 and about 1:2, about 5:1 and about 1:2, about 5:1 and about 2:3, about 4:1 and about 1:2, about 4:1 and about 2:3, about 3:1 and about 1:2, about 3:1 and about 1:2, about 2:1 and about 1:2, about 2:1 and about 2:3, about 1:1 and about 1:2, or about 1:1 and about 2:3.

In yet another aspect, the present invention is directed to a pharmaceutical composition for facilitating weight loss, comprising T₂, T₃ and T₄, and a pharmaceutically acceptable carrier, for example, a sustained release composition.

In yet another aspect, the present invention is directed to a pharmaceutical composition for facilitating weight loss, comprising T₂ and T₃, and a pharmaceutically acceptable carrier, for example, a sustained release composition.

In yet another aspect, the present invention provides a pharmaceutical composition comprising T₂, and a pharmaceutically acceptable excipient, for example, a sustained release formulation.

In any of the foregoing aspects, the pharmaceutical composition includes at least about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mcg of T₃, or, alternatively, between about 5 and about 500 mcg, about 10 and about 400 mcg, about 15 and about 300 mcg, about 20 and about 200 mcg, about 25 and about 150 mcg, or about 50 and about 100 mcg of T₃, and, in particular, between about 25 and about 150 mcg of T₃.

In any of the foregoing aspects, the pharmaceutical composition includes at least about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mcg of T₄, or, alternatively, between between about 5 and about 500 mcg, about 5 and about 400 mcg, about 5 and about 300 mcg, about 10 and about 200 mcg, about 10 and about 150 mcg, or about 50 and about 100 mcg of T₄, and, in particular, between about 25 and about 150 mcg of T₄.

In any of the foregoing aspects, the composition includes at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190 or 200 mcg of T₂, or, alternatively, between about 0.5 and about 200 mcg, about 0.5 and about 150 mcg, about 0.5 and about 100 mcg, about 1 and about 100 mcg, about 1 and about 75 mcg, about 1.5 and about 75 mcg, about 1 and about 50 mcg, about 2 and about 50 mcg, about 1 and about 25 mcg, about 5 and 25 mcg of T₂, about 1 and about 10 mcg, or about 1 and about 5 mcg of T₂.

In a further aspect, the present invention provides a method for facilitating or continuing weight loss in a subject who has undergone a weight loss regimen for at least one month, the method comprising administering to the subject T₂, T₂ in combination with T₃, T₂ in combination with T₃ and T₄, or T₃ in combination with T₄, in an amount effective to increase the level of circulating thyroid hormone in the subject or to decrease the level of rT₃ in the subject to a normal level as present prior to starting the weight loss regimen, thereby facilitating or continuing weight loss in the subject. In yet another aspect, the present invention provides a method for alleviating or preventing a thyroid abnormality in a subject who has undergone a weight loss regimen for at least one month, the method comprising administering to the subject T₂, T₂ in combination with T₃, T₂ in combination with T₃ and T₄, or T₃ in combination with T₄, in an amount effective to increase the level of circulating thyroid hormone in the subject or to decrease the level of rT₃ to a normal level as present prior to starting the weight loss regimen, thereby alleviating or preventing a thyroid abnormality in the subject. For example, the thyroid abnormality is selected from the group consisting of elevated diastolic blood pressure, low blood pressure, elevated lipid levels, coldness, cold extremities, fatigue, insomnia, muscle aches, joint aches, hair loss, constipation, depression and a combination thereof.

### BRIEF DESCRIPTION OF THE DRAWINGS

FIG. 1 depicts hormonal responses to a hypocaloric state with subsequent treatment. Note: Time frames and shapes of response curves are approximated. Levels of all four hormones are normal before the diet (Time A). At time between time points B and D, TSH is dropping slightly to signal the thyroid gland to make less thyroid hormone. This drop is the mechanism to decrease metabolism in the hypocaloric state. At point C, free T₃ begins to drop as rT₃ increases. Also at time point C, different observations have been reported for changes in FT₄: in our experience, FT₄ also drops as indicated on the graph. In the literature, some report no change, whereas others report an increase in FT₄. By the time of the blood sampling (time E), in our experience, patients whose FT₃ and FT₄ have dropped to the low end of normal (with possible above normal rT₃ levels) are those experiencing symptoms of low thyroid. They may have any of the following symptoms: feeling cold, cold hands and feet, elevated diastolic blood pressure secondary to vasoconstriction, fatigue, depression, constipation, hair loss, dry skin, insomnia, as well as others. They have often hit a plateau of weight loss even though they have continued to maintain their hypocaloric diet and exercise program. At point F, we begin treatment with thyroid hormone (for example, T₄ with T₃, T₃ alone, T₃ with T₂ and T₄, or T₂ alone. Between points F and G, FT₃ and rT₃ (and FT₄ if T₄ was administered) are returning to more normal levels, while TSH plummets, often to undetectable levels. At time point H, symptoms of low thyroid are resolved, the patient has no symptoms of hyperthyroidism, and if rT₃ had risen and was treated appropriately, rT₃ decreased.

### DETAILED DESCRIPTION

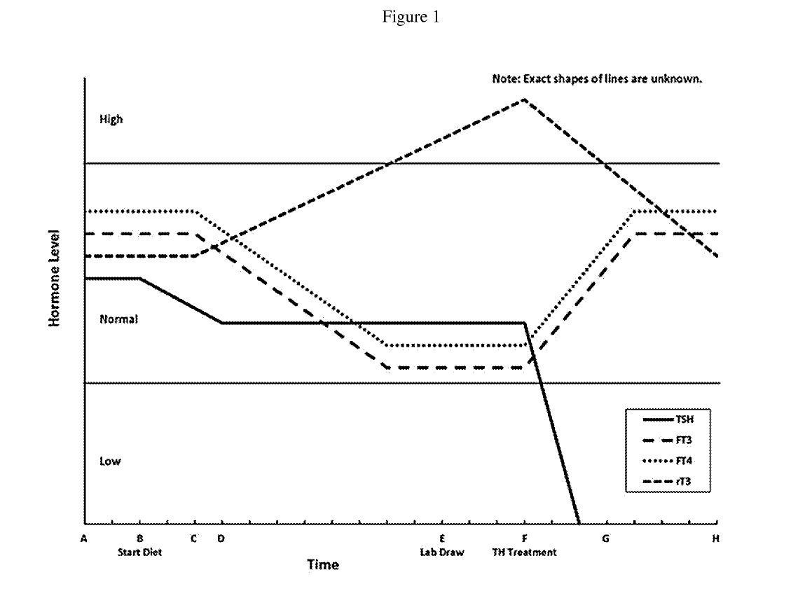

The present invention is based, at least in part, on the finding that the administration of thyroid hormones, for example, T₂ alone, T₃ in combination with T₄, T₃ in combination with T₂ and T₄, or T₃ in combination with T₂, can serve to facilitate weight loss in subjects who are undergoing a weight loss regimen. In particular, subjects who have already experienced weight loss often are unable to continue to lose weight as the body undergoes a famine response, and associated hypothyroidism, resulting from the weight loss regimen. Subjects experience a reduction in circulating thyroid hormones such as T₃, T₂ and T₄ and an increase in rT₃, which blocks the action of the thyroid hormone T₃. Despite maintaining a strict weight loss regimen, the subjects plateau in their efforts to lose weight. However, according to the methods of the present invention (as depicted in FIG. 1), administration of thyroid hormones, for example, T₂ alone, T₃ in combination with T₄, T₃ in combination with T₂ and T₄, or T₃ in combination with T₂, serve to return thyroid levels to normal, so as to allow the body to continue to lose weight.

The present invention is based, at least in part, on the unexpected finding that existing therapy by administration of T₄ alone or T₃ in combination with T₄ at low ratios (e.g., T₃:T₄ at a ratios of less than 1:2) as present in THYROLAR (also known as T₃/T₄ Liotrix) and Armour Thyroid (also known as dessicated thyroid), fails to correct thyroid abnormalities and/or hypothyroidism so as to permit continuing weight loss. Such T₄ based therapy often serves to increase levels of rT₃ and decreases levels of circulating T₃, thereby undermining attempts to counter hypothyroidism and effect continued weight loss. Indeed, the inventors have surprisingly identified that while T₄ is a precursor for both T₃ and RT₃, attempts to counter hypothyroid states by administration of high levels of T₄ often fail because the T₄ results in production of RT₃, not T₃ as desired.

### DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, for example, those characterized by “a” or “an”, shall include pluralities, e.g., one or more biomarkers. In this application, the use of “or” means “and/or”, unless stated otherwise. Furthermore, the use of the term “including,” as well as other forms of the term, such as “includes” and “included”, is not limiting. Also, terms such as “element” or “component” encompass both elements and components comprising one unit and elements and components that include more than one unit unless specifically stated otherwise.

As used herein, the term “thyroid hormone” refers to tyrosine-based hormones produced by the thyroid gland and are primarily responsible for regulation of metabolism. Exemplary thyroid hormones include T₂, T₃ and T₄. “Circulating thyroid hormone,” as used herein, refers to thyroid hormones present in a subject, i.e., in the blood supply of a subject. Circulating thyroid hormone is often referred with a preceding “F”, for example, FT₃ (circulating T₃) or FT₄ (circulating T₄).

As used herein, “T₃” refers to the art recognized thyroid hormone, triiodothyronine (also known as (2S)-2-amino-3-[4-(4-hydroxy-3-iodo-phenoxy)-3,5-diiodo-phenyl]propanoic acid). T₃ has the following structure (C₁₅H₁₂I₃NO₄)

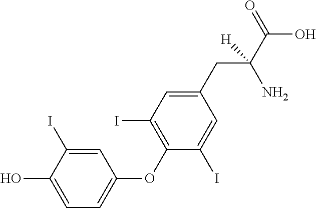 [SMILES: N[C@@H](Cc1cc(I)c(Oc2ccc(O)c(I)c2)c(I)c1)C(=O)O]

T₃ is involved in numerous physiological processes in the body, including growth and development, metabolism, body temperature, and heart rate.

As used herein, the term “T₄” refers to the art recognized thyroid hormone, thyroxine, or 3,5,3′,5′-tetraiodothyronine (also known as (2S)-2-amino-3-[4-(4-hydroxy-3,5-diiododophenoxy)-3,5-diiodophenyl]propanoic acid). T₄ has the following structure (C₁₅H₁₁I₄NO₄):

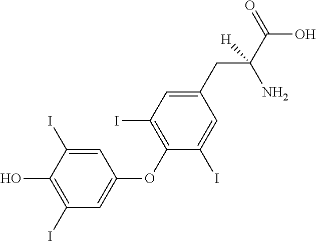 [SMILES: N[C@@H](Cc1cc(I)c(Oc2cc(I)c(O)c(I)c2)c(I)c1)C(=O)O]

T₄ is involved in controlling the rate of metabolic processes in the body and influencing physical development. T₄ is a prohormone and a reservoir for the active thyroid hormone triiodothyronine (T₃), which is about four times more potent. T₄ is converted in the tissues by deiodinases, including thyroid hormone iodine peroxidase (TPO), to T₃.

As used herein, the term “T₂” refers to the thyroid hormone iodothyronine or 3,5-diiodo-1-thyronine. T₂ is involved in stimulating metabolism while simultaneously lowering metabolic efficiency.

As used herein, the term “rT₃” refers to reverse triiodothyronine (reverse T₃). rT₃ has the following chemical structure (C₁₅H₁₂I₃NO₄):

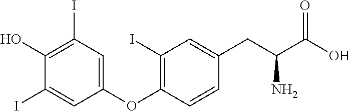 [SMILES: N[C@@H](Cc1ccc(Oc2cc(I)c(O)c(I)c2)c(I)c1)C(=O)O]

rT₃ is an isomer of T₃ and is derived from T₄ through the action of deiodinase. rT₃, unlike T₃, does not stimulate thyroid hormone receptors but instead binds to these receptors, thereby blocking the action of T₃. By blocking T₃, rT₃ is responsible for many of the effects of hypothyroidism and, in turn, inhibits further weight loss in subjects experiencing the famine response.

As used herein, the term “thyroid abnormality” refers to conditions often associated with weight loss and/or hypothyroidism. Thyroid abnormalities include, but are not limited to, elevated diastolic blood pressure, low blood pressure, elevated lipid levels, coldness, cold extremities, fatigue, insomnia, muscle aches, joint aches, hair loss, constipation, depression and a combination thereof.

A “reduced level” of circulating thyroid hormone, e.g., T₃ or T₄, refers to a level of circulating thyroid hormone at lower levels as compared to a suitable control level. In various embodiments, the reduced level is lower than the normal range as generally accepted for the subject, based, for example, on the sex, weight, race and age of the subject. In other embodiments, the reduced level is within the normal range as generally accepted for the subject, but at a lower level within normal range or, lower than at a previous time point for the subject. For example, the reduced level may be within the normal range, but lower than the thyroid hormone level of the subject prior to commencing a weight loss regimen.

The term “known standard level” or “control level” can refer to an accepted or predetermined level of the thyroid hormone which is used to compare the level of the thyroid hormone in a sample derived from a subject. In one embodiment, the control level of the thyroid hormone is the average level of the thyroid hormone samples derived from a population of subjects. For example, the control level can be the average level of thyroid hormone for subjects from a similar population, for example, based on sex, age, weight and race. In other embodiments, the “control” level of thyroid hormone may be determined by determining the level of the respective thyroid hormone biomarker in a subject sample obtained from a subject before commencing a weight loss regimen. As further information becomes available as a result of routine performance of the methods described herein, population-average values for “control” level of thyroid hormones may be used.

As used herein, the term “thyroid hormone therapy” refers to the increase or decrease, preferably, increase, of circulating thyroid hormone in the subject by administration of an agent or a combination of agents, for example, T₃, T₄ and/or T₂. In a particular embodiment, thyroid hormone therapy includes administration of T₃. In another embodiment, thyroid hormone therapy includes administration of T₃ and T₄. In yet another embodiment, thyroid hormone therapy includes administration of T₂. In a further embodiment, thyroid hormone therapy includes administration T₃ and T₂. In yet another embodiment, thyroid hormone therapy includes administration of T₂, T₃ and T₄. In various embodiments, thyroid hormone therapy serves to increase the level of circulating thyroid hormone in the subject to normal levels, for example, the level of the hormone prior to the subject starting a weight loss regimen. For example, thyroid hormone can serve to increase the level of T₃, T₄ and/or T₂ to normal levels. In particular embodiments, thyroid hormone therapy increases thyroid hormone levels to normal levels to counter hypothyroidism, but does not increase levels of thyroid hormone levels to higher than normal levels. In certain embodiments, the methods of the present invention do not increase thyroid hormone levels to higher than the level of thyroid hormone in the subject prior to commencing the weight loss regimen. In other embodiments, the methods of the present invention do not increase thyroid hormone levels to higher than the normal range of thyroid hormone for the subject.

As used herein, the term “weight loss regimen” refers to efforts by a subject to lose weight. Weight loss regimens may include, but are not limited to, reducing caloric intake, a healthier diet, exercise, diet medication, and/or surgical procedures.

As used herein, the term “facilitate weight loss” refers to methods to adjust the subject's hormonal balance to permit effective weight loss. For example, when a subject has experienced weight loss, the subject often experiences a famine response, i.e., a metabolic response which limits the ability of a subject to continue losing weight, despite continued weight loss efforts. By employing the methods of the present invention, i.e., by thyroid hormone therapy as described herein, the methods serve to counter such famine response and facilitate weight loss.

As used herein, the term “subject” or “patient” refers to human and non-human animals, e.g., veterinary patients. The term “non-human animal” includes vertebrates, e.g., mammals, such as non-human primates, mice, rabbits, sheep, dog, cat, horse, cow, or other rodent, ovine, canine, feline, equine or bovine species. In one embodiment, the subject is a human.

The term “sample” as used herein refers to cells, tissues or fluids isolated from a subject, as well as cells, tissues or fluids present within a subject. The term “sample” includes any body fluid (e.g., blood, lymph, cystic fluid, urine), tissue or a cell or collection of cells from a subject, as well as any component thereof, such as a fraction or extract. Other samples, include tears, plasma, serum, cerebrospinal fluid, feces, sputum and cell extracts. In a particular embodiment, the sample is a blood sample derived from the subject.

### Methods for Facilitating or Continuing Weight Loss and Methods for Preventing or Alleviating Thyroid Abnormalities

The present invention provides methods for facilitating weight loss in a subject undergoing a weight loss regimen, by administering an effective amount of at least one thyroid hormone to the subject to increase the level of circulating thyroid hormone in the subject to normal, thereby facilitating weight loss in the subject (as depicted in FIG. 1). The present invention further provides methods for alleviating or preventing a thyroid abnormality (for example, elevated diastolic blood pressure, low blood pressure, elevated lipid levels, coldness, cold extremities, fatigue, insomnia, muscle aches, joint aches, hair loss, constipation and depression) in a subject undergoing a weight loss regimen, by administering an effective amount of at least one thyroid hormone to the subject to increase the level of circulating thyroid hormone in the subject to normal, thereby alleviating or preventing a thyroid abnormality in the subject.

In various embodiments of the foregoing aspects of the invention, the subject has a reduced level of circulating thyroid hormone prior to administration of the thyroid hormone. For example, the subject may have a reduced level of circulating thyroid hormone, such as T₃ and/or T₄, as compared to the level of circulating thyroid hormone prior to the starting the weight loss regimen. Alternatively, the subject may have a reduced level of circulating thyroid hormone, such as T₃ and/or T₄, that while reduced, falls within the normal range. In other embodiments, the subject has a reduced level of thyroid hormone falling below the normal range/control level for the thyroid hormone.

Often such reduced levels of circulating thyroid hormone are associated with the subject having experienced weight loss in the months prior to administration of the thyroid hormone. Such reduced levels constitute reduced metabolic activity often associated with a famine response.

Accordingly, the present invention provides for administration of thyroid hormone to counter the famine response and associated hypothyroidism. For example, the subject may be administered T₃ alone, T₃ in combination with T₄, T₃ in combination with T₂, T₃ in combination with T₂ and T₄, or T₂ alone. In particular embodiments, the subject is administered sustained release formulations of thyroid hormone or combinations of thyroid hormone.

In a particular aspect of the invention, the present invention provides a method for facilitating or continuing weight loss in a subject who has undergone a weight loss regimen for at least one month, by administering thyroid hormone therapy to the subject, wherein the thyroid hormone therapy includes administration of T₂, T₃, T₃ in combination with T₄, T₃ in combination with T₂, or T₃ in combination with T₂ and T₄, to increase the level of circulating thyroid hormone in the subject to a normal level as present prior to starting the weight loss regimen, thereby facilitating or continuing weight loss in the subject.

In another particular aspect of the invention, the present invention provides a method for alleviating or preventing a thyroid abnormality in a subject who has undergone a weight loss regimen for at least one month, by administering thyroid hormone therapy to the subject, wherein the thyroid hormone therapy includes administration of T₂, T₃, T₃ in combination with T₄, T₃ in combination with T₂, or T₃ in combination with T₂ and T₄, to increase the level of circulating thyroid hormone in the subject to a normal level as present prior to starting the weight loss regimen, thereby alleviating or preventing a thyroid abnormality in the subject. In particular embodiments, the thyroid abnormality is selected from the group consisting of elevated diastolic blood pressure, low blood pressure, elevated lipid levels, coldness, cold extremities, fatigue, insomnia, muscle aches, joint aches, hair loss, constipation, depression and a combination thereof.

### Pharmaceutical Compositions

The present invention further provides pharmaceutical compositions for facilitating or continuing weight loss or for alleviating or preventing thyroid abnormalities. The pharmaceutical compositions can include T₃, T₄, T₂, or a combination thereof. In particular embodiments, the pharmaceutical compositions include T₃ or T₂ alone. In other embodiments, the pharmaceutical compositions include T₃, T₂ and T₄. In yet further embodiments, the pharmaceutical compositions include T₃ and T₄. In another embodiment, the pharmaceutical composition include T₃ and T₂.

In one aspect, the present invention is directed to a pharmaceutical composition for facilitating weight loss, including a combination of T₃ and T₄ in a ratio between about 50:1 and about 1:2, about 40:1 and about 1:2, about 30:1 and about 1:2, about 20:1 and about 1:2, about 10:1 and about 1:2, about 5:1 and about 1:2, about 5:1 and about 2:3, about 4:1 and about 1:2, about 4:1 and about 2:3, about 3:1 and about 1:2, about 3:1 and about 1:2, about 2:1 and about 1:2, about 2:1 and about 2:3, about 1:1 and about 1:2, or about 1:1 and about 2:3. In a particular embodiment, the composition further includes T₂.

In another aspect, the present invention is directed to a pharmaceutical composition for facilitating weight loss, including a sustained release formulation of T₃ and T₄. In yet another aspect, the present invention is directed to a pharmaceutical composition for facilitating weight loss, including T₂, T₃ and T₄. In yet another aspect, the present invention is directed to a pharmaceutical composition for facilitating weight loss including T₂ and T₃.

The compositions of the present invention can be incorporated into pharmaceutical compositions (e.g., sustained release, time release and controlled release formulations) suitable for administration. Such compositions typically include at least one thyroid hormone, for example, T₃ alone, T₃ in combination with T₄, T₃ in combination with T₂, T₃ in combination with T₂ and T₄, or T₂ alone, and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules (including, but not limited to soft gelatin capsules) or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

The present invention is further illustrated by the following examples which should not be construed as further limiting. The contents of all references, patents and published patent applications cited throughout this application, as well as the Figures and the Appendix of sequences provided herein, are expressly incorporated herein by reference in their entirety.

### EXAMPLES

### Patient Example 1

The first patient example is a 67 year old female, 5′2″, weight 268 lbs, body mass index (BMI) 49 (nl 19-24; overweight 25-29; obesity≧30). Baseline blood pressure was 110/74, blood sugar (BS) was 153 mg/dL 10 days after her first visit. Her medical problems at baseline included obesity, type 2 diabetes mellitus (DM2), high blood pressure (HTN), fatigue, depression, and asthma. Her medications included glipizide 10 mg daily, amitriptyline 50 mg daily, metoprolol 50 mg daily, furosemide 40 mg daily, potassium chloride, lisinopril 10 mg daily, Advair Discus 500/50 twice daily (bid), and montelukast 10 mg daily. She was started on our comprehensive program. Five months after starting her weight loss treatment, she developed symptoms of hypothyroidism with constipation and fatigue. She had lost 28 lbs by this time and was having difficulty losing more weight even though she was eating less than she was before her weight loss treatment began. At the start of treatment, her FT₄ was 0.9 ng/dL (nl 0.7-1.48). At the time of her low thyroid symptoms, her FT₃ was 2.9 pg/mL (nl 1.71-3.71) and her FT₄ was 0.91 ng/dL (nl 0.7-1.48). She was then treated with thyroid hormone (desiccated thyroid with a ratio of T₄:T₃ of about 4:1) at 60 mg daily for 6 days to acclimate, then increased to 120 mg daily. Since her low thyroid symptoms were only partially improved, her thyroid hormone dose was increased to 150 mg daily 5 months later, with resolution of the constipation and fatigue. After two years on her weight loss treatment, her weight was 231 lbs. Nearly 3 years after the start of her weight loss treatment, while on 150 mg of thyroid hormone, she felt cold and constipated, with another weight loss plateau. We tested for rT₃ at this point. Her labs on the 150 mg dose of desiccated thyroid were: rT₃ 394 pg/mL (nl 90-350), FT₃ 3.94 pg/mL (nl 1.71-3.71), and FT₄ 1.24 ng/dL (nl 0.7-1.48). The task of bringing down her rT₃ was then addressed. Over a period of a few months, her desiccated thyroid hormone was discontinued and instead the patient was given gradually increasing doses of T₃ only, starting at 20 mcg daily and gradually increasing the T₃ to 60 mcg bid. Five months after starting on T₃ with increasing doses, her weight had dropped to 209 lbs. Her BP was 118/66 mmHg with pulse of 74 on lisinopril 10 mg daily, metoprolol 50 mg bid, and furosemide 40 mg daily. Instead of glipizide, she was taking metformin 500 mg bid. She was clinically euthyroid on the T₃ only. Her most recent labs on 60 mcg bid of T₃ were FT₃ 3.8 pg/mL (nl 1.71-3.71), FT₄ 0.64 ng/dL (nl 0.7-1.48) and rT₃ 137 pg/mL (nl 90-350). Four months prior to this visit her BS was 125 mg/dl.

### Comments:

This example makes the important point that three years into the program, with FT₃ above normal and FT₄ in the normal range, the patient had difficulty losing weight and had low thyroid symptoms. We believe that this was due to her elevated level of rT₃. The only way to lower rT₃ in this setting is to avoid giving any T₄ (since T₄ is the source of both T₃ and rT₃) and replace thyroid with T₃ only. Over a period of a few months, both the rT₃ and T₄ will fall while the T₃ should remain in the normal range.

Another point is that there are wide ranges of normal for thyroid tests. Some patients feel optimally well when their thyroid levels are on the high side and some when the thyroid levels are lower in the normal range. For example, a patient with a history of an anxiety disorder or sensitivity to stimulants such as caffeine may feel better when thyroid levels are on the low side of normal. Some require treatment at the high end of normal to eliminate constipation, cold extremities, and/or other symptoms of low thyroid. We think that optimizing symptoms is a good guide along with complete thyroid testing including FT₄, FT₃, and rT₃, especially during caloric restriction. One can optimize symptoms of constipation or diarrhea, feeling too cold or hot, depression or anxiety, or feeling fatigue (which can result from high or low thyroid), etc., by appropriate dosing of T₃ with or without T₄.

### Patient Example 2

The second patient example is a 46 year old female, 5′2″, weight 150 lbs, BMI 28 (nl 18.5-24.9; overweight 25-29.9; obesity≧30), with HTN. Her blood pressure was slightly elevated at 148/85 (on lisinopril 5 mg daily). Her medical problems at baseline included history of Hashimoto's thyroiditis, borderline high TSH, elevated lipids, and HTN. Her medications were lisinopril 5 mg daily, estradiol 2 mg daily, and rofecoxib 25 mg daily for osteoarthritis of her large joints. She was started on our comprehensive program. Thyroid treatment was started as well because of initial low thyroid symptoms of fatigue, constipation, and feeling cold along with a TSH of 3.41 uIU/mL (considered elevated by some, but not all groups who set limits for TSH). Desiccated thyroid was prescribed at 60 mg for six days to adjust to the medication, and then advanced to 120 mg daily. Over a period of several years thyroid hormone dose was reduced to 45 mg as dictated by clinical and lab results, and she was feeling relatively well. Her weight remained between 119 lbs and 130 lbs. Her blood pressure and pulse remained in the normal range (100/60-138/78, pulse 64-72) with no antihypertensive medications. Four years into her treatment, desiccated thyroid became unavailable so she was switched to levothyroxine at 75 mcg daily. Within a few weeks she began to feel tired, cold, and depressed and developed constipation and hair loss. Her FT₃ was 2.3 pg/mL (nl 2.2-4.0), FT₄ 1.07 ng/dL (nl 0.93-1.70), and TSH was 0.91 uIU/mL. Her levothyroxine was increased to 100 mcg daily and T₃ was added at 5 mcg bid. Two months later, her first test of rT₃ was 379 pg/mL (nl 90-350), FT₄ 1.12 ng/dL (nl 0.93-1.70), FT₃ 2.4 pg/mL (nl 2.2-4.0). Over the next 4 months her levothyroxine dose was gradually reduced to zero, and she was placed on gradually increasing doses of T₃ only, ending on 25 mcg bid. Her weight was 125 lbs, BP 122/80 on no medications, and pulse was 72. She no longer suffered from hair loss; her bowels were normal, she felt well, and she stated that for the first time in her life she was not cold. Her lipids were also improved compared to baseline. On this regimen her rT₃ was 193 pg/mL (nl 90-350), FT₃ 2.5 pg/mL (nl 2.2-4.0), and FT₄ 0.67 ng/dL (nl 0.93-1.70).

### Comments:

This patient failed to achieve her optimum health until her rT₃ was mid-normal on T₃ replacement only. In addition, correcting peripheral vasoconstriction results in improved comfort, better sleep, and improved blood pressure, especially diastolic blood pressure. Patients with untreated hypothyroidism may have elevated blood pressure, especially diastolic hypertension, due to peripheral vasoconstriction. After the T₃ treatment to reduce rT₃ levels, a change from T₃ to desiccated thyroid or other ratio of T₄:T₃ dosing would be an option.

### Patient Example 3

Patient 3 is a 43-year old morbidly obese woman in our weight loss program with a history of extreme fatigue. She initially reported fatigue, hair loss, and problems with recall after having been on a sustained release T₃ (T₃SR) 25 mcg t.i.d. regimen. 25 mcg T₄ was added. A few days later, the patient reported “feeling good.” About a month later, while she was still on T₄ 25 mcg q AM and T3 25 mcg t.i.d., lab values were: free T₄ 0.19 ng/dL (normal range 0.93-1.7), free T₃ 4.0 pg/mL (normal range 2.2-4.0), and reverse T₃ 41 pg/mL (normal range 90-350). She subsequently complained of low energy, coldness, cold hands and feet, and constipation. In response to these symptoms, T₄ was discontinued, and desiccated thyroid 60 mg q am was added to replace the T₄ and to try to provide more active thyroid hormones. She reported feeling very well 3 days after this change.

About two months later, the patient was still taking T₃ 25 mcg t.i.d., but reported that she had been out of desiccated thyroid for about a week. She stated that she felt too “up” on the combination of T₃ 25 mcg t.i.d. and desiccated thyroid 60 mg. At this visit, she reported that low thyroid symptoms returned after stopping the desiccated thyroid, specifically, fatigue, coldness, cold hands and feet, and constipation. To try to eliminate the feelings of too much energy, the desiccated thyroid 60 mg was replaced with a sustained released version at the same dose. Labs taken two weeks later showed FT₄ 0.25 ng/dL (normal range 0.93-1.7), FT₃ 4.1 pg/mL (normal range 2.2-4.0), and rT₃ 60 pg/mL (normal range 90-350). About two weeks later, the patient reported that an occasional tremor had been eliminated by alternating between 25 mcg b.i.d. and t.i.d. on consecutive days, thereby decreasing her T₃ dose. We suspected that while the high T₃ dose was needed to produce sufficient T₂ to eliminate coldness, some symptoms of high thyroid were being produced by this T₃ dosage. T₃ was decreased to 25 mcg b.i.d.

T₂ treatment began a few days later at 2 mcg SR q AM. The patient was informed that the treatment was not usual accepted treatment. She was again educated on the symptoms of high thyroid. Two days later, she called to report that her energy levels were normalized.

During her next follow up visit 10 days after the start of T₂ treatment, the patient reported that she “can take a shower and get ready without getting tired . . . Don't have to push myself . . . I feel smarter and able to handle things . . . Sleep like a rock . . . Haven't been craving sugar.” She also reported that the surfaces of her body were now warm and not numb, and that her skin and hair had improved.

Labs showed FT₄ 0.23 ng/dL (normal range 0.93-1.7), FT₃ 3.2 pg/mL (normal range 2.2-4.0), and rT₃ 69 pg/mL (normal range 90-350). So while bringing FT₃ down, the administration of a small dose of T₂ served to make the patient feel generally better.

Over six weeks after starting the T₂ regimen, the patient reported a continued sense of better brain function, regrowth of her hair, firming of fingernails, and significant improvement in her skin, particularly the elimination of dry skin cracking on her feet. She also reported increased light sensitivity with improved close and distant vision, and a resolution of her previous eye fatigue. She was not suffering from any symptoms of low thyroid. Specifically, she denied feeling fatigue, coldness, cold hands or feet and constipation.

### Patient Example 4

Patient #4 is a 36-year old morbidly obese woman in our weight loss program. At the time of her initial visit, she was being treated with T₃ 50 mcg 2 PO q am and 1 PO 8-10 hours later. This regimen had been instituted to bring down a high rT₃ value. She reported feeling cold, hair loss and nail peeling. Labs on the following day showed FT₄ less than 0.1 ng/dL (normal range 0.93-1.7), FT₃ 7.0 pg/mL (normal range 2.2-4.0), and rT₃ less than 15 pg/mL (normal range 90-350). Therefore, despite the high FT₃ and low rT₃, the patient was suffering from low thyroid symptoms. She was advised to decrease the T₃ dosage to 50 mcg, one PO q AM and one PO 8-10 hours later, but neglected to do so. At her next visit over 6 weeks later, she continued to report suffering from fatigue and coldness. Her T₃ was reduced to 50 mcg, one PO q AM and one PO 8-10 hours later. T₂, at 2 mcg one PO q AM and one PO 8-10 hours, was added to her regimen (with appropriate discussion of the rationale and unconventional nature of treatment with T₂).

At her follow up visit nearly two months later, the patient had no complaints of fatigue and reported that only a slight coldness persisted, but even that was much improved. Subsequent labs showed FT₃ returned to a normal value of 2.7 pg/mL (normal range 2.2-4.0).

1. A method for facilitating or continuing weight loss in a subject undergoing a weight loss regimen, the method comprising administering to the subject T₂, T₂ in combination with T₃, T₂ in combination with T₃ and T₄, or T₃ in combination with T₄, in an amount effective to increase the level of circulating thyroid hormone in the subject or to reduce the level of circulating rT₃ hormone to normal, thereby facilitating or continuing weight loss in the subject.

2. A method for alleviating or preventing a thyroid abnormality in a subject undergoing a weight loss regimen, the method comprising administering to the subject T₂, T₂ in combination with T₃, T₂ in combination with T₃ and T₄, or T₃ in combination with T₄, in an amount effective to increase the level of circulating thyroid hormone in the subject or to reduce the level of circulating rT₃ hormone to normal, thereby alleviating or preventing a thyroid abnormality in the subject.

3. The method of claim 2, wherein the thyroid abnormality is selected from the group consisting of elevated diastolic blood pressure, low blood pressure, elevated lipid levels, coldness, cold extremities, fatigue, insomnia, muscle aches, joint aches, hair loss, constipation, depression and a combination thereof.

4. The method of claim 1, wherein the subject has a reduced level of circulating thyroid hormone prior to administration of the thyroid hormone or as compared to the level of circulating thyroid hormone prior to starting the weight loss regimen.

5. (canceled)

6. The method of claim 4, wherein the subject has a reduced level of circulating T₃, circulating T₄ or a combination thereof.

7. The method of claim 1, wherein the subject has elevated levels of circulating rT₃ prior to administration of the thyroid hormone.

8. The method of claim 1, wherein the subject has experienced weight loss prior to administration of the thyroid hormone.

9-10. (canceled)

11. The method of claim 1, wherein the weight loss regimen comprises dieting, exercise, weight loss surgery and/or appetite control medication.

12. (canceled)

13. The method of claim 1, wherein the subject is administered T₂ alone.

14-26. (canceled)

27. The method of claim 1, wherein at least one of T₂, T₃ and T₄ is administered in a sustained release composition.

28. The method of claim 1, wherein the combination of T₂ and T₃; T₂, T₃ and T₄; or T₃ and T₄ are provided in a single formulation.

29. The method of claim 28, wherein the formulation is a sustained release formulation.

30-31. (canceled)

32. A pharmaceutical composition for facilitating weight loss, comprising T₃ and T₄; T₂, T₃ and T₄; T₂ and T₃; or T₂; and a pharmaceutically acceptable carrier.

33. The pharmaceutical composition of claim 32, wherein the composition is a sustained release composition.

34. (canceled)

35. The pharmaceutical composition of claim 32, comprising T₂, T₃ and T₄ and a pharmaceutically acceptable carrier.

36. The pharmaceutical composition of claim 35, wherein the composition is a sustained release composition.

37. The pharmaceutical composition of claim 32, comprising T₂ and T₃; and a pharmaceutically acceptable carrier.

38. The pharmaceutical composition of claim 37, wherein the composition is a sustained release composition.

39-44. (canceled)

45. The pharmaceutical composition of claim 32, comprising T₂, and a pharmaceutically acceptable excipient.

46. The pharmaceutical composition of claim 45, wherein the composition is a sustained release formulation.

47-55. (canceled)
